(19) United States
(12) Patent Application Publication
(10) Pub. No.: US 2019/0071716 A1
(43) Pub. Date: Mar. 7, 2019

(54) BODILY FLUID ENRICHMENT

(71) Applicant: GENETICS RESEARCH, LLC, D/B/A ZS GENETICS, INC., Wakefield, MA (US)

(72) Inventor: Anthony P. Shuber, Northbridge, MA (US)

(21) Appl. No.: 16/177,784

(22) Filed: Nov. 1, 2018

Related U.S. Application Data
(63) Continuation-in-part of application No. 16/018,926, filed on Jun. 26, 2018.
(60) Provisional application No. 62/672,217, filed on May 16, 2018.
(60) Provisional application No. 62/526,091, filed on Jun. 28, 2017.

(51) Int. Cl. C12Q 1/6827; C12Q 1/6806; C12Q 1/6809; C12Q 1/6811; C12Q 1/686

(52) U.S. Cl. CPC C12Q 1/6827; C12Q 1/6806; C12Q 1/686; C12Q 1/6811; C12Q 1/6809

(57) ABSTRACT

The invention provides methods for binding to and protecting target nucleic acid directly from plasma without the need for certain complex sample preparation steps, using catalytically active Cas endonuclease. The catalytically active Cas endonucleases, along with their sequence-specific guide RNAs, may be introduced directly into the plasma sample, where the catalytically active Cas endonucleases bind to ends of a target nucleic acid. The target nucleic acid is thus isolated or enriched in a sequence-specific manner. The target nucleic acid may then be subject to any suitable detection or analysis assay, such as amplification or sequencing. The bound catalytically active Cas proteins prevent exonuclease from digesting the target nucleic acid in a plasma sample leaving only the target nucleic acid substantially present in the enriched sample.

### CROSS REFERENCE TO RELATED APPLICATIONS

This application is a continuation-in-part of U.S. Non-Provisional application Ser. No. 16/018,926, filed Jun. 26, 2018, which claims the benefit of, and priority to, U.S. Provisional Application No. 62/526,091, filed Jun. 28, 2017, and U.S. Provisional Application No. 62/672,217, filed May 16, 2018, the contents of each of which are incorporated by reference.

### TECHNICAL FIELD

The invention relates to molecular genetics.

### BACKGROUND

When testing for diseases, such as cancer, physicians rely on liquid and tissue biopsy from a subject. After obtaining the liquid and tissue biopsy, which may be a painful process for the subject, the liquid or tissue biopsy must be analyzed. However, existing analysis methods require expensive and time-consuming sample preparation procedures, kits, and reagents.

For example, in a liquid biopsy, a blood sample is taken from a patient and may be centrifuged to remove whole blood cells, leaving plasma or serum that includes cell-free DNA (cfDNA). Typically, the sample must be subject to a sample preparation protocol before any genetic analysis is performed. For example, laboratory technicians use a commercially-available kit to aliquot the serum through a series of steps that use proteinase solutions to digest away proteins, lysis buffers to dissociate vesicles and other lipid fragments, and cleaning and suspension buffers. In some protocols, the resultant mixture is washed through a membrane within a column under vacuum after which the cfDNA is eluted from the column with a specialty wash buffer. The entire process can require hours or more and the use of expensive kits. Some companies offer specialty instruments to aid in automating some of those steps. The kits and instruments are expensive, but theoretically provide isolated cfDNA for analysis.

### SUMMARY

The present invention provides methods for capturing target nucleic acid directly from bodily fluid samples without the need for significant sample preparation steps or kits. Methods of the invention use Cas endonuclease to bind target nucleic acid sequences of interest. Since Cas endonuclease binds and protects specific targets in vivo, and a bodily fluid sample has qualities similar to cytoplasm, as such Cas binds and protects targets in the bodily fluid sample without the need for significant sample preparation. Furthermore, since Cas endonuclease binds to and protects specific targets in vivo, and a nucleic acid sample spiked into a solution is less complex than cytoplasm or a bodily fluid, Cas endonuclease binds to and protects nucleic acid in the solution.

Surprisingly, catalytically inactive Cas endonuclease fails to remain bound to targets in a plasma sample when exposed to exonuclease, despite the ability of catalytically inactive Cas endonuclease to bind to and protect a target in a solution containing target nucleic acid. As such, in a preferred embodiment, a catalytically active Cas endonuclease captures and protects target nucleic acid directly from bodily fluid samples without the need for significant sample preparation steps or kits. In another embodiment, catalytically inactive Cas endonuclease captures and protects target nucleic acid in a sample that is not plasma. In yet another embodiment, catalytically inactive Cas endonuclease captures, but does not protect target nucleic acid directly from plasma without the need for significant sample preparation steps or kits.

The Cas endonuclease is provided with one or more guide RNAs that bind to target nucleic acid that includes or flanks a locus of interest, such as a locus of a known cancer-associated mutation or a specific genetic allele of clinical interest. The catalytically active Cas endonuclease binds to and protects target nucleic acid even when a variant is only present as a small fraction of a plasma sample, where the catalytically inactive Cas endonuclease binds to and protects target nucleic acid binds to and protects target nucleic acid even when a variant is only present as a small fraction of a sample that is not plasma. Thus, methods of the invention are useful when analyzing nucleic acid present in low abundance in a sample, and where the sample is plasma, catalytically active Cas endonuclease is useful in the methods disclosed herein. In a preferred embodiment, once captured by the Cas endonuclease, the target may then be analyzed or sequenced to report and use the genetic information, e.g., to detect or monitor cancer.

Catalytically active Cas endonucleases, along with their sequence-specific guide RNAs (gRNA), are introduced directly into the plasma sample. The catalytically active Cas endonucleases may be introduced as part of the sample collection, or added into collection tubes containing the plasma sample. Catalytically inactive Cas endonucleases, along with their sequence-specific guide RNAs (gRNA), are introduced directly into a sample that is not plasma. The catalytically inactive Cas endonucleases may be introduced as part of the sample collection, or added into collection tubes containing the sample that is not plasma. In any sample type, the gRNA mediates binding of the Cas endonucleases to a target nucleic acid of interest, such as tumor DNA fragment harboring a clinically significant mutation.

In a preferred embodiment, catalytically active Cas endonuclease binds to the target nucleic acid in a plasma sample and the target nucleic acid is then protected when enriched relative to other materials in the sample by elimination of non-target nucleic acid using, for example, exonucleases. Enrichment methods may be used alone or in combination with other enrichment methods. As a non-limiting example, exonuclease digestion may be used alone, or may be used before elution of catalytically active Cas endonuclease bound targets. The target nucleic acid may be subject to any suitable detection or analysis assay, such as amplification or sequencing.

In another embodiment, catalytically inactive or catalytically active Cas endonuclease binds to and protects the target nucleic acid in a sample that is not plasma and the target nucleic acid is then protected when enriched relative to other materials in the sample by elimination of non-target nucleic acid using, for example, exonucleases. Enrichment methods may be used alone or in combination with other enrichment methods. As a non-limiting example, exonuclease digestion may be used alone, or may be used before elution of catalytically inactive or catalytically active Cas endonuclease bound targets. The target nucleic acid may be subject to any suitable detection or analysis assay, such as amplification or sequencing.

Methods and related kits described herein are useful to detect the presence of a target nucleic acid, such as a variant, in a sample. Due to the nature by which a protein, such as a Cas complex, binds nucleic acid, methods may be used even where the target is present only in very small quantities, e.g., even as low as 0.01% frequency of variant fragments among normal fragments in a sample (i.e., where about 50 copies of a circulating tumor DNA fragment harboring a mutation are present among about 500,000 unrelated fragments of similar size). Thus, methods of the invention may have particular applicability in discovering very rare, yet clinically important, information, such as mutations that are specific to a tumor and may be used to detect specific mutations among cell-free DNA, such as tumor mutations among circulating tumor DNA.

In a preferred method, CRISPR/Cas systems and associated guide RNAs are introduced to a plasma sample. When used according to methods of the invention, catalytically active Cas endonuclease, and not catalytically inactive Cas endonuclease (i.e., dCas) will bind to a target nucleic acid consistently via a guide RNA and will protect that target (i.e., stay bound), thereby allowing the target to be obtained out of the plasma sample by elimination of the non-target sequence. In certain aspects, the invention provides methods for detecting a target nucleic acid in a plasma sample. Methods include obtaining a plasma sample from a subject, introducing catalytically active Cas endonuclease and guide RNA into the plasma, binding the catalytically active Cas endonuclease to ends of a target nucleic acid, protecting the target nucleic acid with catalytically active Cas endonuclease when negatively enriching the target nucleic acid and isolating the target nucleic acid from the plasma sample.

In another method of the invention, CRISPR/Cas systems and associated guide RNAs are introduced to a sample that is not plasma. When used according to methods of the invention, Cas endonuclease will bind to a target nucleic acid consistently via a guide RNA and will protect that target (i.e., stay bound), thereby allowing the target to be obtained out of the non-plasma sample by elimination of the non-target sequence. In certain aspects, the invention provides methods for detecting a target nucleic acid in a non-plasma sample. Methods include obtaining a non-plasma sample from a subject, introducing Cas endonuclease and guide RNA into the sample, binding the Cas endonuclease to ends of a target nucleic acid, negatively enriching the target nucleic acid and isolating the target nucleic acid from the sample.

In yet another method of the invention, CRISPR/Cas systems and associated guide RNAs are introduced to a plasma sample. When used according to methods of the invention, catalytically inactive Cas endonuclease (i.e., dCas or dCas9) will bind to a target nucleic acid in a plasma sample via a guide RNA thereby allowing the target to be obtained out of the plasma sample by isolation of the target sequence. In certain aspects, the invention provides methods for detecting a target nucleic acid in a plasma sample. Methods include obtaining a non-plasma sample from a subject, introducing Cas endonuclease and guide RNA into the sample, binding the Cas endonuclease to ends of a target nucleic acid, negatively enriching the target nucleic acid and isolating the target nucleic acid from the sample.

The nucleic acid may be any naturally-occurring or artificial nucleic acid. The nucleic acid may be DNA, RNA, hybrid DNA/RNA, peptide nucleic acid (PNA), morpholine and locked nucleic acid (LNA), glycol nucleic acid (GNA), threose nucleic acid (TNA), or Xeno nucleic acid. The RNA may be a subpopulation of RNA, such as mRNA, tRNA, rRNA, miRNA, or siRNA. Preferably the nucleic acid is DNA.

The target or feature of interest may be any feature of a nucleic acid. The feature may be a variant. For example and without limitation, the feature may be a mutation, insertion, deletion, substitution, inversion, amplification, duplication, translocation, or polymorphism. The feature may be a nucleic acid from an infectious agent or pathogen. For example, the nucleic acid sample may be obtained from an organism, and the feature may contain a sequence foreign to the genome of that organism.

The target nucleic acid may be from a sub-population of nucleic acid within the nucleic acid sample. For example, the target nucleic acid may contain cell-free DNA, such as cell-free fetal DNA or circulating tumor DNA. In some embodiments, the sample includes plasma from the subject and the target nucleic acid is cell-free DNA (cfDNA). The plasma may be maternal plasma and the target may be of fetal DNA. In certain embodiments, the sample includes plasma from the subject and the target is circulating tumor DNA (ctDNA). In certain embodiments, the sample does not include plasma, but includes target nucleic acid suspended in a solution, such as a buffer solution. In some embodiments, the sample includes at least one circulating tumor cell from a tumor and the target is tumor DNA from the tumor cell. In some embodiments, the target nucleic acid is complementary DNA (cDNA), which is made by reverse transcribing RNA. In some embodiments, detecting cDNA is a way to detecting target RNA.

The target nucleic acid may be from any source of nucleic acid. In preferred embodiments, the target nucleic acid is from a bodily fluid sample from a human. In a preferred embodiment, the bodily fluid sample is plasma. In preferred embodiments, the bodily fluid sample is a liquid or bodily fluid from a subject, such as bile, blood, plasma, serum, sweat, saliva, urine, feces, phlegm, mucus, sputum, tears, cerebrospinal fluid, synovial fluid, pericardial fluid, lymphatic fluid, semen, vaginal secretion, products of lactation or menstruation, amniotic fluid, pleural fluid, rheum, vomit, or the like. In preferred embodiments, the bodily fluid sample is a blood sample, serum sample, plasma sample, urine sample, saliva sample, semen sample, feces sample, phlegm sample, or liquid biopsy. The sample may be a tissue sample from an animal, such as skin, conjunctiva, gastrointestinal tract, respiratory tract, vagina, placenta, uterus, oral cavity or nasal cavity. The sample may be a liquid biopsy or a tissue biopsy.

In some embodiments, obtaining the sample includes obtaining a bodily fluid sample from a subject in a collection tube. In a non-limiting example, the bodily fluid is blood and the collection tube is centrifuged to isolate serum or plasma from blood cells. The catalytically active Cas endonuclease is introduced into the serum or plasma. In an embodiment, the catalytically active Cas endonuclease is introduced into the serum or plasma as a ribonucleoprotein (RNP) in which the endonuclease is complexed with the guide RNA. Preferably, the guide RNA includes at least two single guide RNA molecules that each complex with a catalytically active Cas endonuclease and guide the catalytically active Cas endonuclease to hybridize to one of the target, wherein the target includes a loci know to harbor a disease-associated variant. The method includes elimination of the non-target sequence by exposing the sample to, for example, exonuclease.

In some other embodiments, obtaining the sample includes obtaining a bodily fluid sample from a subject in a collection tube. In a non-limiting example, the bodily fluid is blood and the collection tube is centrifuged to isolate serum or plasma from blood cells. The catalytically inactive Cas endonuclease is introduced into the serum or plasma. In an embodiment, the catalytically inactive Cas endonuclease is introduced into the serum or plasma as a ribonucleoprotein (RNP) in which the catalytically inactive Cas endonuclease is complexed with the guide RNA. Preferably, the guide RNA includes at least two single guide RNA molecules that each complex with a catalytically inactive Cas endonuclease and guide the catalytically active Cas endonuclease to hybridize to, but not protect, one of the targets, wherein the target includes a loci know to harbor a disease-associated variant.

In another non-limiting example, the target nucleic acid is suspended in a buffer solution. The catalytically inactive Cas endonuclease is introduced into the solution. In an embodiment, the catalytically inactive Cas endonuclease is introduced into the solution as a ribonucleoprotein (RNP) in which the catalytically inactive Cas endonuclease is complexed with the guide RNA. Preferably, the guide RNA includes at least two single guide RNA molecules that each complex with a catalytically inactive Cas endonuclease and guide the catalytically active Cas endonuclease to hybridize to and protect one of the targets, wherein the target includes a loci know to harbor a disease-associated variant. The method includes elimination of the non-target sequence by exposing the sample to, for example, exonuclease.

In other embodiments, the method may include separating the Cas endonuclease-bound target nucleic acid from some or all of the unbound nucleic acid. For example, the method may include binding the Cas endonuclease-bound target nucleic acid to a particle. The particle may include magnetic or paramagnetic material. The method may include applying a magnetic field to the sample. The particle may include an agent that binds to a Cas endonuclease bound to an end of the target nucleic acid. The agent may an antibody or fragment thereof. The method may include chromatography, applying the sample to a column, or gel electrophoresis. The method may include separating the Cas endonuclease-bound target nucleic acid from some or all of the unbound nucleic acid by size exclusion, ion exchange, or adsorption.

Each of the Cas endonucleases binds to a nucleic acid in a sequence-specific manner. The Cas endonuclease may be complexed with a nucleic acid that guides the protein to an end of the nucleic acid. For example, the catalytically active Cas endonuclease is in a complex with one or more guide RNAs in a plasma sample.

The target nucleic acid may be detected by any means known in the art. For example and without limitation, the target nucleic acid may be detected by DNA staining, spectrophotometry, sequencing, fluorescent probe hybridization, fluorescence resonance energy transfer, optical microscopy, or electron microscopy. Detecting the target nucleic acid may include identifying a variant in the target nucleic acid. Identifying the variant may include sequencing the nucleic acid (e.g., on a next-generation sequencing instrument), allele-specific amplification, and hybridizing a probe to the nucleic acid.

Methods of the invention may include amplifying the target nucleic acid to yield amplicons. Methods may further include sequencing the target nucleic acid to produce sequence reads and analyzing the sequence reads to provide genetic information of the subject. Methods may include analyzing the target nucleic acid to describe one or more variants in the subject.

In some embodiments, the target nucleic acid includes a mutation specific to a tumor. The target nucleic acid may be present at no more than about 0.01% of cell-free DNA in the plasma or serum. By methods herein, catalytically active Cas endonuclease binds to, cleaves and protects the target nucleic acid and the target nucleic acid is then isolated or enriched from the serum or plasma. In other embodiments, catalytically inactive Cas endonuclease binds the target nucleic acid and the target nucleic acid is then isolated or positively enriched from the serum or plasma.

Certain methods may further include detecting the target nucleic acid (e.g., by amplification, sequencing, probe hybridization, digital PCR, etc.). For example, detecting the target nucleic acid may include hybridizing the target nucleic acid to a probe or to a primer for a detection or amplification step, or labelling the target nucleic acid with a detectable label. Because the Cas endonucleases are used to bind to the target in a sequence-specific manner, and thereby enrich and isolate for a specific variant, detecting the presence of the nucleic acid may be useful to report the presence of the variant in a subject from whom the sample is obtained. In multiplexed embodiments, a panel or any number of specific variants is assayed for through use of steps of the methods and the results may provide a description or count of tumor mutations detected from the target nucleic acid in the bodily fluid sample.

Furthermore, methods of the invention include negative enrichment in a plasma or serum sample with catalytically active Cas endonuclease, and not catalytically inactive Cas (i.e., dCas). As an example, catalytically active Cas endonuclease may be provided with one or more guide RNAs that bind to a target nucleic acid and flank a loci of interest, such as a locus of a known cancer-associated mutation or a specific genetic allele of clinical interest. The catalytically active Cas endonuclease binds to, cleaves and protects variant-containing nucleic acid even when the variant is only present as a small fraction of the plasma sample. The catalytically active Cas endonuclease bound to the target nucleic acid prevent exonuclease from digesting the target nucleic acid and, after incubation with exonuclease, the only nucleic acid substantially present in the sample is the target nucleic acid. The target nucleic acid is thus isolated or negatively enriched in a sequence-specific manner. The target nucleic acid may then be subject to any suitable detection or analysis assay such as amplification or sequencing.

In a preferred method, CRISPR/Cas systems using catalytically active Cas (and not dCas) and guide RNAs specific for a variant are introduced to the sample under conditions such that nucleic acid containing the variant or mutation is protected from exonuclease digestion while non-target nucleic acid is digested by an exonuclease. When used according to methods of the invention, catalytically active Cas endonuclease, and not catalytically inactive Cas endonuclease, will bind to a target consistently via a guide RNA and will protect that target (i.e., stay bound) for at least long enough that a promiscuous exonuclease can be reliably used to digest unbound, non-target nucleic acid in a plasma sample. By protection of the target with digestion of the non-target, a sample is effectively negatively enriched for the target, and those remaining target fragments are captured, stored, isolated, preserved, detected, sequenced, or otherwise assayed with success that would be unobtainable without methods of the invention.

In certain aspects, the invention provides a method for detecting a target nucleic acid. The method includes obtaining a serum or plasma sample from a subject, introducing catalytically active Cas endonuclease (e.g., Cas9), and not dCas, and guide RNA into the serum or plasma, and binding the catalytically active Cas endonuclease to ends of a target nucleic acid. Unbound nucleic acid is digested from the sample by introducing exonuclease while the catalytically active Cas endonuclease prevents the exonuclease from digesting the target nucleic acid, thereby enriching the sample for the target nucleic acid. The target nucleic acid may then be isolated from the enriched sample by amplification, size fractionation, or hybrid capture. Methods may include inactivating the exonuclease (e.g., by heating) prior to the isolating step. Preferably, two catalytically active Cas endonucleases bind to ends of the target nucleic acid and prevent the exonuclease from digesting the target nucleic acid.

### BRIEF DESCRIPTION OF THE DRAWINGS

FIG. 1 shows a table of the inputs and the dilution amounts used in Example 1 described herein. Dilution 11 is at 3× concentration from previous experiments because the experiment uses 3× as much input DNA volume in the reaction. The copies per ul of stock, copies per ul in 50 ul reaction, amount of previous dilution (ul), plasma, and total volume (ul) are indicated.

FIG. 2 shows a table of the dilutions used in Example 1. For the percent of plasma in the final reaction, the percent of plasma in 2× sample, plasma dilution (ul), and tris dilution (ul) are shown in the table.

FIG. 3 shows a graph of the qPCR results after amplification from the post-cutting dilutions described in Example 1.

FIG. 4 shows the tabulated qPCR results from Example 1. Percent plasma, use of a Streck tube, amount of no Cas9 present, amount of Cas9 present, and percent cutting are indicated.

FIG. 5 shows a chart of the binding efficiency from Example 1, particularly showing the relationship between percent cleavage and percent plasma. In particular, the percent cleavage is shown as a function of the amount or percent of plasma in the cutting reaction. Results are shown for samples with no tube and samples using a Streck tube.

FIG. 6 shows a chart of the detection signal in plasma from Example 1, particularly showing the relationship between qPCR signal and percent plasma. In particular, the percent detection of no plasma in the sample is shown as a function of the percent plasma in the cutting reaction. Results are shown for samples with no tube and sample using a Streck tube.

FIG. 7 provides the reaction summary of the formation of Cas9-guide RNA complexes for use in samples comprised of 820 bp amplicon in 10 mM Tris, pH 7.5 (250 ng, 2.5 uL, 100 ng/uL).

FIG. 8 depicts the results of an exemplary agarose gel electrophoresis of the activity of biotinylated dCas9, dCas9, and Cas9 complexed with guide RNA CFTR F2 on the target amplicon in Tris.

FIG. 9 provides the composition of the control samples and the samples containing CFTR amplicon before adding 1.06 uL of the control or the Cas9 complex.

FIG. 10 provides the reaction summary of the formation of Cas9-guide RNA complexes for use in samples comprised of 820 bp amplicon in plasma. 1.06 uL of the complex was used per sample reaction.

FIG. 11 depicts the results of an exemplary agarose gel electrophoresis of the activity of dCas9 and Cas9 complexed with guide RNA CFTR F2 or biotinylated guide RNA CFTR F2 on the target amplicon in Tris.

FIG. 12 depicts the results of an exemplary agarose gel electrophoresis of the activity of dCas9 and Cas9 complexed with guide RNA CFTR F2 or biotinylated guide RNA CFTR F2 on the target amplicon in human plasma.

### DETAILED DESCRIPTION

Methods of the invention provide for the protection of a target nucleic acid, in a sequence-specific manner, directly from samples without the need for complex sample preparation. Preferred embodiments include obtaining a bodily fluid sample from a subject. Certain embodiments of the invention provide a method for detecting a target nucleic acid in the bodily fluid sample.

Surprisingly, catalytically inactive Cas endonuclease (i.e., dCas9) fails to remain bound to targets in a plasma sample when exposed to exonuclease, despite ability to bind to and protect a target in a solution comprising target nucleic acid and a buffer. As such, in a preferred embodiment, a catalytically active Cas endonuclease captures and protects target nucleic acid directly from plasma samples without the need for significant sample preparation steps or kits. In another embodiment, catalytically inactive Cas endonuclease captures and protects target nucleic acid in a solution. In yet another embodiment, catalytically inactive Cas endonuclease captures, but does not protect target nucleic acid directly from a plasma sample.

Methods of the invention include introducing the Cas endonuclease and guide RNA directly into the bodily fluid sample. Embodiments of the invention use Cas endonuclease proteins that are originally encoded by genes that are associated with clustered regularly interspaced short palindromic repeats (CRISPR) in bacterial genomes. A CRISPR-associated (Cas) endonuclease may be introduced directly into the bodily fluid sample. In a preferred embodiment the Cas endonuclease introduced directly into the plasma sample is Cas9.

The Cas endonucleases bind to ends of a target nucleic acid. The target nucleic acid is thus isolated or enriched in a sequence-specific manner. The enriched target nucleic acid is further enriched by digesting other, unbound nucleic acids present in the sample with exonuclease. The bound catalytically active Cas endonucleases prevent the exonuclease from digesting the target nucleic acid, thereby leaving the only the target nucleic acid substantially present in the sample. Surprisingly, dCas9 endonuclease dead (i.e., dCas9), does not protect the target nucleic acid in plasma when exonuclease is used to digest non-target nucleic acid. As such, only catalytically active Cas endonuclease, and preferably, Cas9, may be used to bind to and protect target nucleic acid when performing negative enrichment by digesting non-target nucleic acid sequences with exonuclease in a plasma sample. The target nucleic acid is thus negatively enriched in a sequence-specific manner. The target nucleic acid may then be subject to any suitable detection or analysis assay such as amplification or sequencing.

Preferably, the Cas endonuclease is complexed with a guide RNA that targets the Cas endonuclease to a specific sequence. Any suitable catalytically active Cas endonuclease or homolog thereof may be used in plasma to capture and protect target nucleic acid. A Cas endonuclease may be Cas9 (e.g., spCas9), Cpf1 (aka Cas12a), C2c2, Cas13, Cas13a, Cas13b, e.g., PsmCas13b, LbaCas13a, LwaCas13a, AsCas12a, others, modified variants thereof, and similar proteins or macromolecular complexes. In other embodiments, any suitable catalytically inactive or catalytically active Cas endonuclease or homolog thereof may be used in a non-plasma sample to capture and protect target nucleic acid. A Cas endonuclease may be Cas9 (e.g., spCas9), a catalytically inactive Cas (dCas9), Cpf1 (aka Cas12a), C2c2, Cas13, Cas13a, Cas13b, e.g., PsmCas13b, LbaCas13a, LwaCas13a, AsCas12a, others, modified variants thereof, and similar proteins or macromolecular complexes. The Cas13 proteins may be preferred where the target includes RNA. A Cas endonuclease/guide RNA complex includes a first Cas endonuclease and a first guide RNA. In the depicted embodiment, the complex comprises the catalytically active Cas endonuclease being introduced into the plasma as a ribonucleoprotein (RNP) in which the catalytically active Cas endonuclease is complexed with the guide RNA. In another embodiment depicted herein, the complex comprises either the catalytically active or the catalytically inactive Cas endonuclease being introduced into the non-plasma sample as a ribonucleoprotein (RNP) in which the catalytically active or the catalytically inactive Cas endonuclease is complexed with the guide RNA. In either embodiment, the Cas endonuclease will bind to the target. The target may then be isolated or enriched, allowing for detection of the target. If the target is present in a plasma sample, then the target may then be enriched by digesting non-target nucleic acid with exonuclease when the target nucleic acid is bound to the catalytically active Cas endonuclease.

The proteins that bind to ends of the target nucleic acid may be any proteins that bind to a nucleic acid in a sequence-specific manner. The protein may be a programmable nuclease. For example, the protein may be a CRISPR-associated (Cas) endonuclease, zinc-finger nuclease (ZFN), transcription activator-like effector nuclease (TALEN), or RNA-guided engineered nuclease (RGEN). Programmable nucleases and their uses are described in, for example, Zhang, 2014, “CRISPR/Cas9 for genome editing: progress, implications and challenges”, Hum Mol Genet 23 (R1):R40-6; Ledford, 2016. CRISPR: gene editing is just the beginning, Nature. 531 (7593): 156-9; Hsu, 2014, Development and applications of CRISPR-Cas9 for genome engineering, Cell 157(6):1262-78; Boch, 2011, TALEs of genome targeting, Nat Biotech 29(2):135-6; Wood, 2011, Targeted genome editing across species using ZFNs and TALENs, Science 333(6040):307; Carroll, 2011, Genome engineering with zinc-finger nucleases, Genetics Soc Amer 188(4):773-782; and Urnov, 2010, Genome Editing with Engineered Zinc Finger Nucleases, Nat Rev Genet 11(9):636-646, each incorporated by reference.

In some embodiments, the protein used in a non-plasma sample may be a catalytically inactive form of a nuclease, such as a programmable nuclease described above. The protein may be a transcription activator-like effector (TALE). The protein may be complexed with a nucleic acid that guides the protein to an end of the target nucleic acid. For example, the protein may be a catalytically inactive Cas endonuclease in a complex with one or more guide RNAs. In preferred embodiments, the protein is a catalytically inactive Cas endonuclease, or homologs thereof.

In certain embodiments, the plasma sample includes cfDNA from a subject. The plasma sample is exposed to a first catalytically active Cas endonuclease/guide RNA complex that binds to a target nucleic acid (e.g., a mutation of interest) in a sequence-specific fashion. In some embodiments, the complex binds to a mutation in a sequence-specific manner. A segment of the nucleic acid, i.e., the target nucleic acid, is protected by introducing the first catalytically active Cas endonuclease/guide RNA complex and a second catalytically active Cas endonuclease/guide RNA complex that also binds to the nucleic acid. In preferred embodiments of the method, the guide RNA comprises at least two guide RNA molecules that each complex with a catalytically active Cas endonuclease and guide the catalytically active Cas endonuclease to hybridize to one target nucleic acid, wherein the target nucleic acid includes a loci know to harbor a cancer-associated mutation. The unprotected nucleic acid is digested. For example, one or more exonucleases may be introduced that promiscuously digest unbound, unprotected nucleic acid. Any suitable exonuclease may be used. Suitable exonucleases include, for example, Lambda exonuclease, RecJf, Exonuclease III, Exonuclease I, Exonuclease T, Exonuclease V, Exonuclease VII, T5 Exonuclease, and T7 Exonuclease, most of which are available from New England Biolabs (Ipswich, Mass.). While the exonucleases act, the target nucleic acid is protected by the bound catalytically active Cas endonuclease complexes and survives the digestion step intact. In a preferred embodiments, Cas9 is utilized.

The described steps including the digestion by the exonuclease leave a reaction product that includes principally only the variant segment of nucleic acid, as well as any spent reagents, catalytically active Cas endonuclease complexes, exonuclease, nucleotide monophosphates, and pyrophosphate as may be present.

In certain embodiments, the exonuclease is deactivated. For example, exonuclease may be deactivated by following the manufacturer's instructions e.g., by heating to 90 degrees for a few minutes. After digestion, a positive selection step may be performed which may include, for example, amplification of the target nucleic acid by known methods or selection by an affinity assays.

The nucleic acid may be any naturally-occurring or artificial nucleic acid. The nucleic acid may be DNA, RNA, hybrid DNA/RNA, peptide nucleic acid (PNA), morpholine and locked nucleic acid (LNA), glycol nucleic acid (GNA), threose nucleic acid (TNA), or Xeno nucleic acid. The RNA may be a subpopulation of RNA, such as mRNA, tRNA, rRNA, miRNA, or siRNA. Preferably the nucleic acid is DNA.

The target or feature of interest may be any feature of a nucleic acid. The feature may be a variant. For example and without limitation, the feature may be a mutation, insertion, deletion, substitution, inversion, amplification, duplication, translocation, or polymorphism. The feature may be a nucleic acid from an infectious agent or pathogen. For example, the nucleic acid sample may be obtained from an organism, and the feature may contain a sequence foreign to the genome of that organism.

The target nucleic acid may be from a sub-population of nucleic acid within the nucleic acid sample. For example, the target nucleic acid may contain cell-free DNA, such as cell-free fetal DNA or circulating tumor DNA. In some embodiments, the sample includes plasma from the subject and the target nucleic acid is cell-free DNA (cfDNA). The plasma may be maternal plasma and the target may be of fetal DNA. In certain embodiments, the sample includes plasma from the subject and the target is circulating tumor DNA (ctDNA). In some embodiments, the sample includes at least one circulating tumor cell from a tumor and the target is tumor DNA from the tumor cell. In some embodiments, the target nucleic acid is complementary DNA (cDNA), which is made by reverse transcribing RNA. In some embodiments, detecting cDNA is a way to detecting target RNA.

The target nucleic acid may be from any source of nucleic acid. In preferred embodiments, the target nucleic acid is from a bodily fluid sample from a human. In preferred embodiments, the bodily fluid is plasma. In preferred embodiments, the bodily fluid sample is a liquid or bodily fluid from a subject, such as bile, blood, plasma, serum, sweat, saliva, urine, feces, phlegm, mucus, sputum, tears, cerebrospinal fluid, synovial fluid, pericardial fluid, lymphatic fluid, semen, vaginal secretion, products of lactation or menstruation, amniotic fluid, pleural fluid, rheum, vomit, or the like. In preferred embodiments, the bodily fluid sample is a blood sample, serum sample, plasma sample, urine sample, saliva sample, semen sample, feces sample, phlegm sample, or liquid biopsy. The sample may be a tissue sample from an animal, such as skin, conjunctiva, gastrointestinal tract, respiratory tract, vagina, placenta, uterus, oral cavity or nasal cavity. The sample may be a liquid biopsy or a tissue biopsy.

The method optionally includes detecting the target nucleic acid (which may harbor the variant or mutation). Any suitable technique may be used to detect the target nucleic acid. For example, detection may be performed using DNA staining, spectrophotometry, sequencing, fluorescent probe hybridization, fluorescence resonance energy transfer, optical microscopy, electron microscopy, others, or combinations thereof. Detecting the target nucleic acid may indicate the presence of the mutation in the subject (i.e., a patient), and a report may be provided describing the variant or mutation in the patient.

In an embodiment of the invention, a sample may contain a mutant fragment of DNA, a wild-type fragment of DNA, or both. A locus of interest is identified where a mutation may be present proximal to, or within, a protospacer adjacent motif (PAM). When the wild-type fragment is present, it may contain a wild-type allele at a homologous location in the fragment, also proximal to, or within, a PAM. A guide RNA is introduced to the sample that has a targeting portion complementary to the portion of the mutant fragment that includes the mutation. When a Cas endonuclease is introduced, it will form a complex with the guide RNA and bind to the mutant fragment but not to the wild-type fragment. The first Cas endonuclease/guide RNA complex includes a guide RNA with a targeting region that binds to the mutation but that does not bind to other variants at a loci of the mutation. The described methodology may be used to target a mutation that is proximal to a PAM, or it may be used to target and detect a mutation in a PAM, e.g., a loss-of-PAM or gain-of-PAM mutation.

The described methodology may be used to target a mutation that is proximal to a PAM, or it may be used to target and detect a mutation in a PAM, e.g., a loss-of-PAM or gain-of-PAM mutation. The PAM is typically specific to, or defined by, the Cas endonuclease being used. For example, for Streptococcus pyogenes Cas9, the PAM includes NGG, and the targeted portion includes the 20 bases immediately 5′ to the PAM. As such, the targetable portion of the DNA includes any twenty-three consecutive bases that terminate in GG or that are mutated to terminate in GG. Such a pattern may be found to be distributed over ctDNA at such frequency that the potentially detectable mutations are abundant enough as to be representative of mutations over the tumor DNA at large. In such cases, Cas9, and not dCas9, may be used to detect mutations from a tumor in a plasma sample when using endonuclease to enrich the targetable portion of tumor DNA. Moreover, methods may be used to determine a number of mutations over the representative, targetable portion of tumor DNA. Since the targetable portion of the genome is representative of the tumor DNA overall, the number of mutations may be used to infer a mutational burden for the tumor.

A feature of the method is that a specific mutation may be detected by a technique that includes detecting only the presence or absence of a fragment of DNA in a plasma sample, and it need not be necessary to sequence DNA from a subject to describe variants. Methods of the invention use catalytically active Cas endonuclease, preferably Cas9, for protection at one or both ends of DNA segments. The gRNA selects for a known variant on one end. A positive selection may be performed to positively select out the bound, target nucleic acid. If the gRNA does not find the mutation, no protection is provided and the molecule may be digested, e.g. in negative enrichment, and the remaining molecules are either counted or sequenced. Methods are well suited for the analysis of samples in which the target of interest is extremely rare, and particularly for the analysis of maternal plasma or serum (e.g., for fetal DNA).

Methods are useful for the isolation of intact DNA fragments of any arbitrary length and may preferably be used in some embodiments to isolate or enrich for arbitrarily long fragments of DNA, e.g., tens, hundreds, thousands, or tens of thousands of bases in length or longer in plasma. Long, isolated, intact fragments of DNA may be analyzed by any suitable method such as simple detection (e.g., via staining with ethidium bromide) or by single-molecule sequencing. It is noted that the catalytically active Cas9/gRNA complexes may be subsequently or previously labeled using standard procedures. The complexes may be fluorescently labeled, e.g., with distinct fluorescent labels such that detecting involves detecting both labels together (e.g., after a dilution into fluid partitions). Preferred embodiments of the detection do not require PCR amplification and therefore significantly reduces cost, reaction time, and sequence bias associated with PCR amplification. Sample analysis can also be performed by a number of approaches, such as next generation sequencing (NGS), etc. However, many analytical platforms may require PCR amplification prior to analysis. Therefore, preferred embodiments of analysis of the reaction products include single molecule analysis that avoids the requirement of amplification.

Kits and methods of the invention are useful with methods disclosed in U.S. Provisional Patent Application 62/526,091, filed Jun. 28, 2017, for POLYNUCLEIC ACID MOLECULE ENRICHMENT METHODOLOGIES and U.S. Provisional Patent Application 62/519,051, filed Jun. 13, 2017, for POLYNUCLEIC ACID MOLECULE ENRICHMENT METHODOLOGIES, both incorporated by reference.

The target nucleic acid may be detected, sequenced, or counted. Where a plurality of fragments are present or expected, the fragment may be quantified, e.g., by qPCR.

The target nucleic acid may further be isolated or detected by any suitable method in order to separate the target segment from other nucleic acids in the sample. In preferred embodiments, the isolation or detection method includes separating the Cas9-bound target nucleic acid from some or all of the unbound nucleic acid by exonuclease digestion. In other embodiments, the isolation or detection method may include binding the protein-bound target nucleic acid to a particle. In some embodiments catalytically active Cas9-bound target is bound to a particle before or after digestion. The particle may include magnetic or paramagnetic material. The isolation or detection method may include applying a magnetic field to the sample. The particle may include an agent that binds to a protein bound to an end of the target nucleic acid. The agent may an antibody or fragment thereof. The isolation or detection method may include chromatography. The isolation or detection method may include applying the sample to a column. The isolation or detection method may include separating the protein-bound target nucleic acid from some or all of the unbound nucleic acid by size exclusion, ion exchange, or adsorption. The isolation or detection method may include gel electrophoresis.

Embodiments of the invention may include detecting the target nucleic acid and optionally providing a report describing a mutation as present in the patient. The mutation-containing fragments may be detected by a suitable assay, such as sequencing, gel electrophoresis, or a probe-based assay. The detection of the isolated segment of the target nucleic acid may be done by sequencing. The digestion provides a reaction product that includes principally only the target nucleic acid, as well as any spent reagents, catalytically active Cas endonuclease complexes, exonuclease, nucleotide monophosphates, or pyrophosphate as may be present. The reaction product may be provided as an aliquot (e.g., in a micro centrifuge tube such as that sold under the trademark EPPENDORF by Eppendorf North America (Hauppauge, N.Y.) or glass cuvette). The reaction product aliquot may be disposed on a substrate. For example, the reaction product may be pipetted onto a glass slide and subsequently combed or dried to extend the fragment across the glass slide. The reaction product may optionally be amplified. Optionally, adaptors are ligated to ends of the reaction product, which adaptors may contain primer sites or sequencing adaptors. The presence of the segment in the reaction product aliquot may then be detected using an instrument.

The target nucleic acid may be detected by any means known in the art. For example and without limitation, the target nucleic acid may be detected by DNA staining, spectrophotometry, sequencing, fluorescent probe hybridization, fluorescence resonance energy transfer, optical microscopy, or electron microscopy. Detecting the nucleic acid may include identifying a variant in the nucleic acid. Identifying the variant may include sequencing the nucleic acid (e.g., on a next-generation sequencing instrument), allele-specific amplification, and hybridizing a probe to the nucleic acid. Methods of DNA sequencing are known in the art and described in, for example, Peterson, 2009, Generations of sequencing technologies, Genomics 93(2):105-11; Goodwin, 2016, Coming of age: ten years of next-generation sequencing technologies, Nat Rev Genet 17(6):333-51; and Morey, 2013, A glimpse into past, present, and future DNA sequencing, Mol Genet Metab 110(1-2):3-24, each incorporated by reference. Other methods of DNA detection are known in the art and described in, for example, Xu, 2014, Label-Free DNA Sequence Detection through FRET from a Fluorescent Polymer with Pyrene Excimer to SG, ACS Macro Lett 3(9):845-848, incorporated by reference.

One method for detection is gel electrophoresis. Gel electrophoresis allows separation of molecules based on differences in their sizes and is thus useful when the target nucleic acid is larger than other nucleic acids, for example the residual nucleic acids left from a digestion step. In a preferred embodiment, gel electrophoresis is performed on the Cas9 bound target nucleic acid after exonuclease digestion of the non-target nucleic acid. Methods of gel electrophoresis are known in the art and described in, for example, Tom Maniatis; E. F. Fritsch; Joseph Sambrook. “Chapter 5, protocol 1”. Molecular Cloning—A Laboratory Manual. 1 (3rd ed.). p. 5.2-5.3. ISBN 978-0879691363; and Ninfa, Alexander J.; Ballou, David P.; Benore, Marilee (2009). Fundamental laboratory approaches for biochemistry and biotechnology. Hoboken, N.J.: Wiley. p. 161. ISBN 0470087668, the contents of which are incorporated herein by reference.

One method for detection of protein-bound nucleic acids is immunomagnetic separation. Magnetic or paramagnetic particles are coated with an antibody that binds the protein bound to the segment, and a magnetic field is applied to separate particle-bound segment from other nucleic acids. Methods of immunomagnetic purification of biological materials such as cells and macromolecules are known in the art and described in, for example, U.S. Pat. No. 8,318,445; Safarik and Safarikova, Magnetic techniques for the isolation and purification of proteins and peptides, Biomagn Res Technol. 2004; 2:7, doi: 10.1186/1477-044X-2-7, the contents of each of which are incorporated herein by reference. The antibody may be a full-length antibody, a fragment of an antibody, a naturally occurring antibody, a synthetic antibody, an engineered antibody, or a fragment of the aforementioned antibodies. Alternatively or additionally, the particles may be coated with another protein-binding moiety, such as an aptamer, peptide, receptor, ligand, or the like.

Chromatographic methods may be used for detection. In such methods, the bodily fluid sample is applied to a column, and the target nucleic acid is separated from other nucleic acids based on a difference in the properties of the target nucleic acid and the other nucleic acids. Size exclusion chromatography is useful for separating molecules based on differences in size and thus is useful when the segment is larger than other nucleic acids, for example the residual nucleic acids left from a digestion step. Methods of size exclusion chromatography are known in the art and described in, for example, Ballou, David P.; Benore, Marilee; Ninfa, Alexander J. (2008). Fundamental laboratory approaches for biochemistry and biotechnology (2nd ed.). Hoboken, N.J.: Wiley. p. 129. ISBN 9780470087664; Striegel, A. M.; and Kirkland, J. J.; Yau, W. W.; Bly, D. D.; Modern Size Exclusion Chromatography, Practice of Gel Permeation and Gel Filtration Chromatography, 2nd ed.; Wiley: NY, 2009, the contents of each of which are incorporated herein by reference.

Ion exchange chromatography uses an ion exchange mechanism to separate analytes based on their respective charges. Thus, ion exchange chromatography can be used with the proteins bound to the target nucleic acid impart a differential charge as compared to other nucleic acids. Methods of ion exchange chromatography are known in the art and described in, for example, Small, Hamish (1989). Ion chromatography. New York: Plenum Press. ISBN 0-306-43290-0; Tatjana Weiss, and Joachim Weiss (2005). Handbook of Ion Chromatography. Weinheim: Wiley-VCH. ISBN 3-527-28701-9; Gjerde, Douglas T.; Fritz, James S. (2000). Ion Chromatography. Weinheim: Wiley-VCH. ISBN 3-527-29914-9; and Jackson, Peter; Haddad, Paul R. (1990). Ion chromatography: principles and applications. Amsterdam: Elsevier. ISBN 0-444-88232-4, the contents of each of which are incorporated herein by reference.

Adsorption chromatography relies on difference in the ability of molecule to adsorb to a solid phase material. Larger nucleic acid molecules are more adsorbent on stationary phase surfaces than smaller nucleic acid molecules, so adsorption chromatography is useful when the target nucleic acid is larger than other nucleic acids, for example the residual nucleic acids left from a digestion step. Methods of adsorption chromatography are known in the art and described in, for example, Cady, 2003, Nucleic acid purification using microfabricated silicon structures. Biosensors and Bioelectronics, 19:59-66; Melzak, 1996, Driving Forces for DNA Adsorption to Silica in Perchlorate Solutions, J Colloid Interface Sci 181:635-644; Tian, 2000, Evaluation of Silica Resins for Direct and Efficient Extraction of DNA from Complex Biological Matrices in a Miniaturized Format, Anal Biochem 283:175-191; and Wolfe, 2002, Toward a microchip-based solid-phase extraction method for isolation of nucleic acids, Electrophoresis 23:727-733, each incorporated by reference.

Certain preferred embodiments include obtaining a blood, plasma, or serum sample from a patient. Preferably, plasma is obtained from a patient. The blood, plasma, or serum may include cfDNA and thus also include ctDNA among the cfDNA. Specific sequences of the ctDNA are isolated or enriched and analyzed or detected to detect or report genetic information from the subject, such as a presence or count of certain tumor mutations. Methods of the invention include introduce Cas endonucleases (or catalytically inactive homologs thereof such as dCas9) directly into serum or plasma. The Cas endonucleases are complexed with guide RNAs that include targeting portions specific for a target nucleic acid. In the plasma or serum, the catalytically active Cas endonuclease (e.g., Cas9 and not dCas9) complexes bind to ends of the target and protects it when exonuclease is introduced to digest unbound nucleic acid into monomers and fragments too small for further meaningful detection, sequencing, or amplification. Importantly, dCas9 complexes are only able to bind to the ends of the target, but cannot protect it when exonuclease is introduced in a plasma sample. However, dCas9 complexes are able to bind to and protect target nucleic acid in a non-plasma sample when exonuclease is introduced.

Embodiments of the invention provide for treatment of a sample. For example, a blood sample may be obtained from a patient. The sample may be collected in any suitable blood collection tube such as the collection tube sold under the trademark VACUTAINER by BD (Franklin Lakes, N.J.). In certain embodiments, the collection tube comprises an EDTA collection tube, and Na-EDTA collection tube or the collection tube sold under the trademark CELL-FREE DNA BCT by Streck, Inc. (La Vista, Nebr.), sometimes referred to in the art as a Streck tube. Use of a Streck tube stabilizes nucleated blood cells and prevents the release of genomic DNA into the sample. This facilitates the collection of sample that includes cell-free DNA.

The sample may be centrifuged to generate a sample that includes a pellet of blood cells and a supernatant, which contains serum or plasma. Serum is the liquid supernatant of whole blood that is collected after the blood is allowed to clot and centrifuged. Plasma is produced when the process includes an anticoagulant. To collect serum, blood is collected in tubes. After collection, the blood is allowed to clot by leaving it undisturbed at room temperature (about 15-30 minutes). The clot is removed by centrifuging, e.g., at 1,000-2,000×g for 10 minutes in a refrigerated centrifuge. The resulting supernatant is designated serum and may be transferred to a clean polypropylene tube using a Pasteur pipette. For plasma, blood is collected into commercially available anticoagulant-treated tubes e.g., EDTA-treated (lavender tops), citrate-treated (light blue tops), or heparinized tubes (green tops), followed by centrifugation to collect the supernatant. The supernatant is preferably transferred to a fresh tube, away from the pellet, which may be discarded. Particularly where the collection tube included an anticoagulant, the transfer should give a good separation of the plasma from the whole blood cells. After transfer, the sample includes plasma or serum, which includes cfDNA.

In an exemplary embodiment, serum or plasma is transferred from a centrifuge tube to a new tube, complexes comprising Cas9, and not dCas9, and guide RNA are added, and the mixture is incubated and exonuclease is introduced to digest unbound, non-target DNA, and then the exonuclease may be deactivated (e.g., by heat). A positive selection may then follow (e.g., amplification or an affinity assay) to positively select out the bound, target nucleic acid.

In another exemplary embodiment, plasma or serum is removed from the centrifuge tube (the supernatant) and transferred into a new tube. Appropriate buffers/reagents are added to modify a chemical environment to promote binding of Cas endonuclease (catalytically active or inactive) to the target nucleic acid. For example, pH can be adjusted, as may temperature, salinity, or co-factors present. The Cas complexes are added and allowed to incubate. For example, amplification or an affinity may be performed to positively select out the bound, target nucleic acid. An exonuclease may optionally be added, which ablates all free, non-target nucleic acid. The target may be positively selected such as by amplification or an affinity assay after exonuclease digestion of the non-target nucleic acid.

Methods may include detection or isolation of circulating tumor cells (CTCs) from a blood sample. Cytometric approaches use immunostaining profiles to identify CTCs. CTC methods may employ an enrichment step to optimize the probability of rare cell detection, achievable through immune-magnetic separation, centrifugation, or filtration. Cytometric CTC technology includes the CTC analysis platform sold under the trademark CELLSEARCH by Veridex LLC (Huntingdon Valley, Pa.). Such systems provide semi-automation and proven reproducibility, reliability, sensitivity, linearity and accuracy. See Krebs, 2010, Circulating tumor cells, Ther Adv Med Oncol 2(6):351-365 and Miller, 2010, Significance of circulating tumor cells detected by the CellSearch system in patients with metastatic breast colorectal and prostate cancer, J Oncol 2010:617421-617421, both incorporated by reference.

Certain embodiments of the invention may provide a kit. The kit preferably includes reagents and materials useful for performing methods of the invention. For example, the kit may include one or more guide RNA that, taken in pairs, are designed to flank cancer-associated mutations. The kit may include one or more guide RNAs that are mutation specific and only hybridize to target that includes a mutation. The kit may include a Cas endonuclease or a nucleic acid encoding a Cas endonuclease such as a plasmid. In a preferred embodiment, the kit includes Cas9 plasmid and exonuclease. The kit may include reagents for adjusting conditions such as pH, salinity, co-factors, etc., to promote binding or activity of Cas endonuclease in the bodily fluid sample, such as plasma or serum. The kit may further include instructional materials for performing methods of the invention, and components of the kit may be packaged in a box suitable for shipping or storage. Preferably, the kit contains one or more collection tubes, such as a blood collection tube.

The Cas endonuclease/guide RNA complexes can be designed to bind to variants of clinical significance, such as a mutation specific to a tumor. Preferably, Cas9/guide RNA complexes are designed to bind to variants of clinical significance, such as a mutation specific to a tumor directly in a plasma sample. When a mutation is thus detected, a report may be provided to, for example, describe the mutation in a patient or a subject. Thus, certain embodiments may comprise providing a report. The report preferably includes a description of the mutation in the subject (e.g., a patient). The method for detecting rare nucleic acid may be used in conjunction with a method of describing mutations (e.g., as described herein). Either or both detection processes may be performed over any number of loci in a patient's genome or preferably in a patient's tumor DNA. As such, the report may include a description of a plurality of structural alterations, mutations, or both in the patient's genome or tumor DNA. As such, the report may give a description of a mutational landscape of a tumor.

Knowledge of a mutational landscape of a tumor may be used to inform treatment decisions, monitor therapy, detect remissions, or combinations thereof. For example, where the report includes a description of a plurality of mutations, the report may also include an estimate of a tumor mutation burden (TMB) for a tumor. It may be found that TMB is predictive of success of immunotherapy in treating a tumor, and thus methods described herein may be used for treating a tumor.

Methods of the invention thus may be used to detect and report clinically actionable information about a patient or a tumor in a patient. For example, the method may be used to provide a report describing the presence of the genomic alteration in a genome of a subject. Additionally, protecting a segment of DNA, and optionally digesting unprotected DNA, provides a method for isolation or enrichment of DNA fragments, i.e., the protected segment. It may be found that the described enrichment techniques are well-suited to the isolation/enrichment of arbitrarily long DNA fragments, e.g., thousands to tens of thousands of bases in length or longer.

Long DNA fragment targeted enrichment, or negative enrichment, creates the opportunity of applying long read platforms in clinical diagnostics. Negative enrichment may be used to enrich “representative” genomic regions that can allow an investigator to identify “off rate” when performing CRISPR Cas9 experimentation, as well as enrich for genomic regions that would be used to determine TMB for immuno-oncology associated therapeutic treatments. In such applications, the negative enrichment technology is utilized to enrich large regions (>50 kb) within the genome of interest using Cas9, and not dCas9 in a plasma sample.

By the described methods, a bodily fluid sample can be assayed for a mutation using a technique that is inexpensive, quick, and reliable. Methods of the invention are conducive to high throughput embodiments, and may be performed, for example, in droplets on a microfluidic device, to rapidly assay a large number of aliquots from a sample for one or any number of genomic structural alterations.

### EXAMPLE 1

The cutting efficiency of amplicons by Cas9 in plasma is shown by experiment. Results from the experiment indicated that Cas proteins bind to expected cognate targets under guide RNA guidance in plasma or serum. In particular, Cas9 was tested for cutting activity in plasma in an experimental protocol.

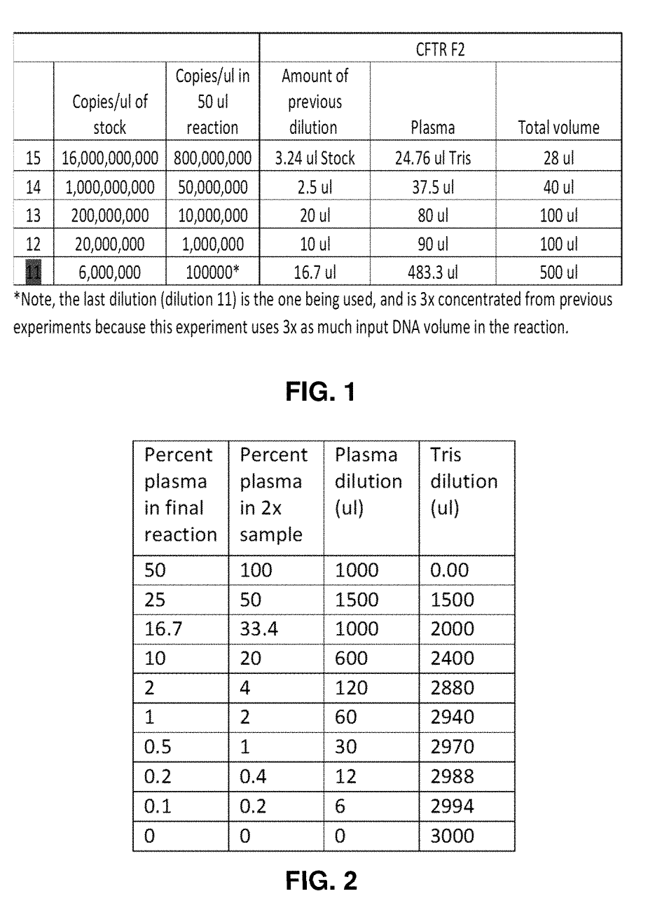

Plasma samples were placed in Streck tubes and in standard tubes. The experiments used an 800 bp amplicon from the cystic fibrosis transmembrane receptor gene. Dilutions were made of CFTR F2 800 bp into plasma with 5 million copies per reaction total (FIG. 1). The percent plasma in reaction after dilution was 50%, 25%, 16.7%, 10%, 2%, 1%, 0.5%, 0.2%, 0.1%, and 0% (FIG. 2).

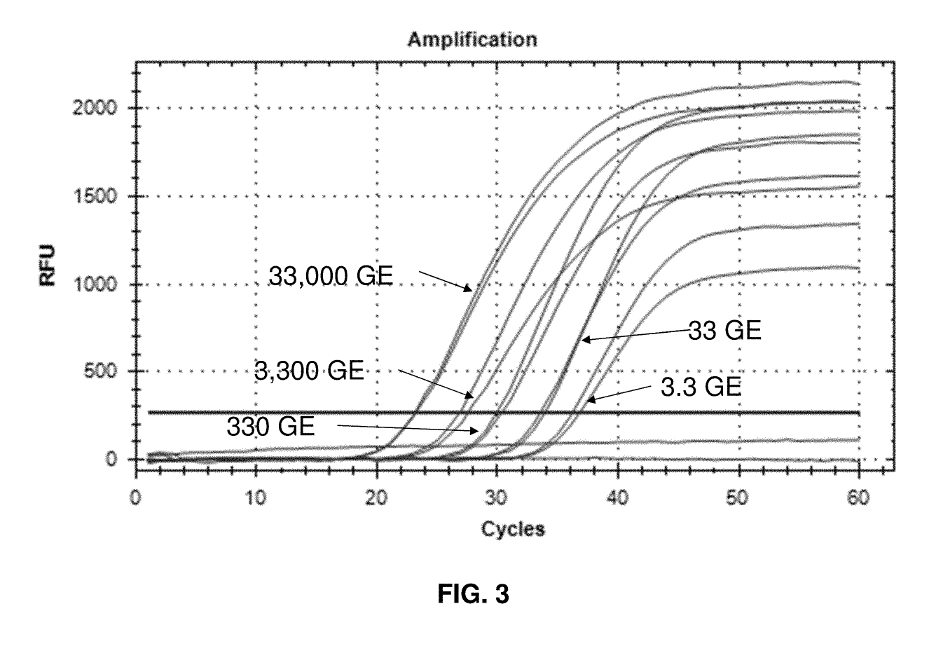

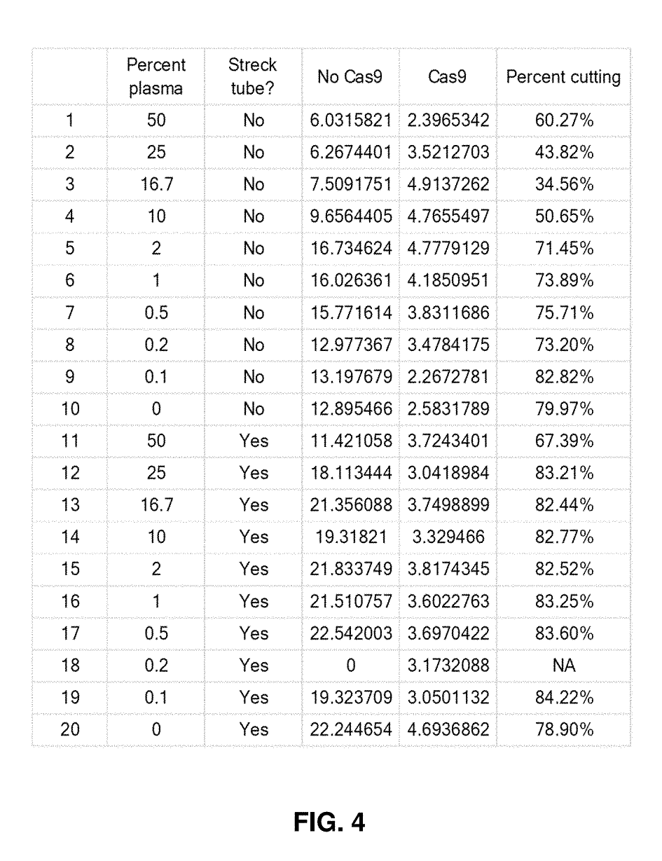

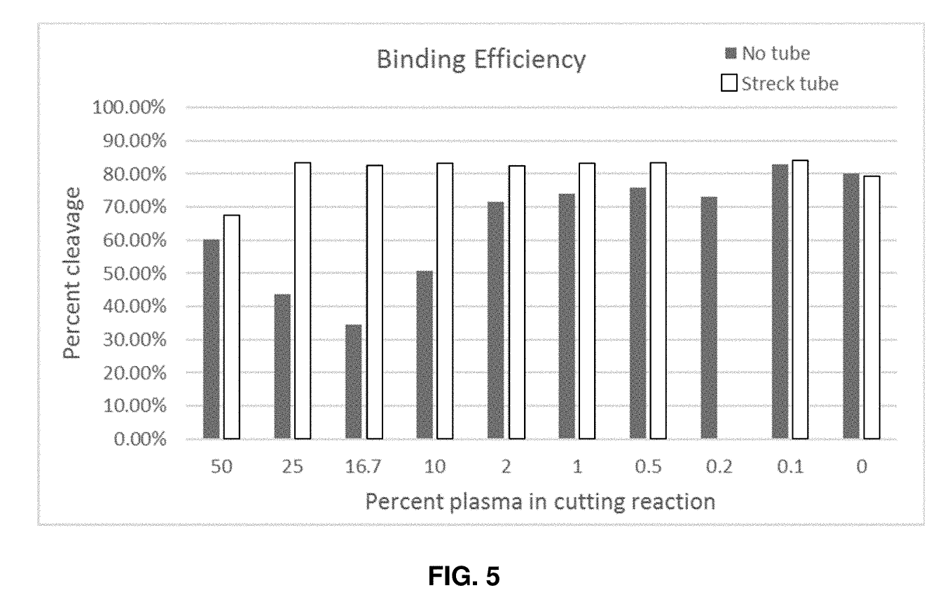

Cas9 with guide RNA was added and allowed to cut. qPCR was then used to probe across the cut site. For qPCR, samples were diluted 1/100, and then 5 ul were used per 20 ul reaction. The qPCR results were analyzed from amplifying, post-cutting, from dilutions (FIGS. 3 and 4). The qPCR results indicated cleavage as a function of plasma amount (FIG. 5). For example, every replicate in a Streck tube demonstrated greater than 60% cutting efficiency by Cas9 in the CFTR amplicon. Cas9 exhibited detectable cutting, even in standard, non-Streck tubes.

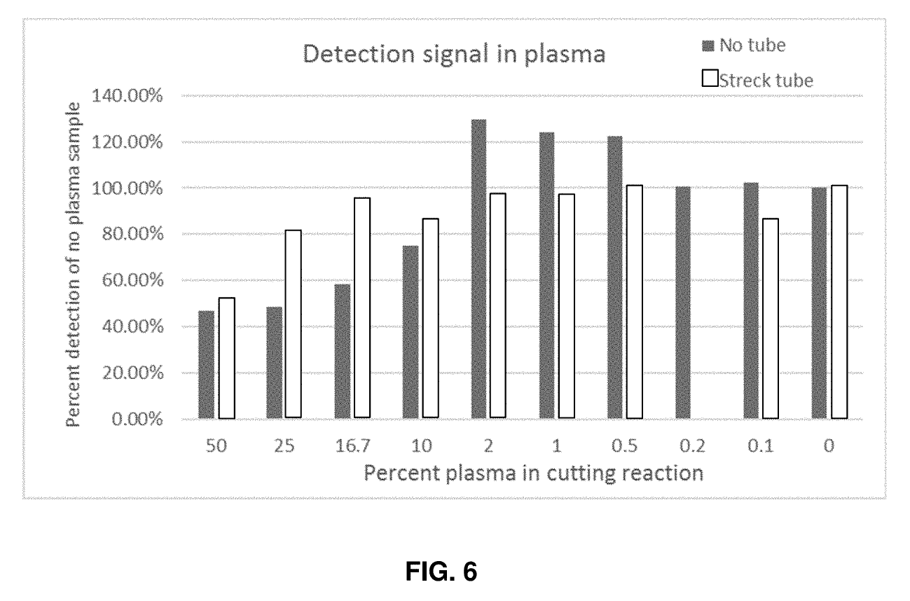

The results also indicated a relationship between the qPCR signal and percent plasma (FIG. 6). For example, the data show Cas9 exhibits detectable cutting in Na-EDTA plasma. For the reactions performed in straight plasma, cutting efficiency in 2% plasma or lower resembled no plasma cutting efficiency (82.82% for in plasma compared to 79.97% in no plasma). For the reactions performed in plasma incubated in a Streck tube, the cutting efficiency in 25% plasma or lower resembled no-plasma cutting efficiency (83.14% compared to 78.90%). Further, there was 60-67% cutting for the 50% plasma samples. In 50% plasma, CRISPR/Cas9 complexes retained 75% activity. Results of the data show that Cas endonuclease and homologs thereof bind to target DNA under guidance of guide RNA in plasma.

### EXAMPLE 2

The binding and protection activity of both Cas9 and dCas9 is shown by this experiment. Results from this experiment indicate dCas proteins bind to the target under RNA guidance when the target is present in a solution of Tris background and Cas9 buffer. In particular, dCas9 was tested for protection activity in the absence of cleavage.

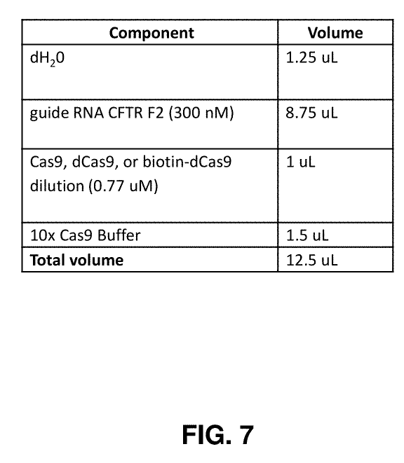

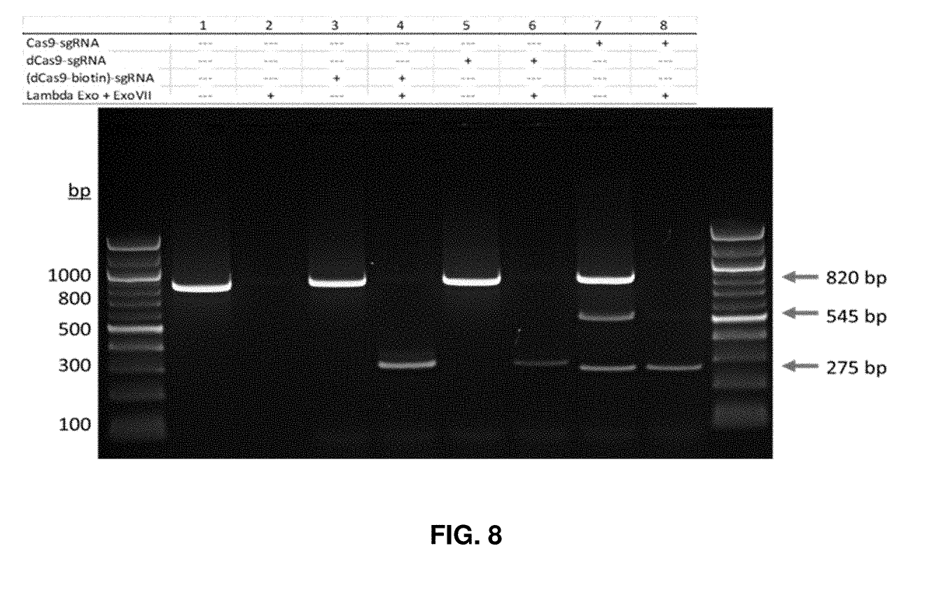

The experiments utilized a 820 bp amplicon from the cystic fibrosis transmembrane receptor gene. Samples were made consisting of the 820 bp CFTR amplicon added to 10 mM Tris (250 ng, 2.5 uL, 100 ng/uL), pH 7.5. The Cas9-guide RNA complexes were then made consisting of: 1.25 uL of distilled water, 1.5 uL of Cas9 buffer (10×), 1.0 uL of dCas9, dCas9 biotinylated, or Cas9 (0.77 uM) complexed with 8.75 uL of CFTR F2 guide RNA (300 nM) (FIG. 7). The control “complexes” made consisted of 11 uL of water and 1.5 uL of 10× Cas9 buffer (FIG. 8, Lanes 1 and 2). The Cas9-guide RNA reagents were added directly to the CFTR-Tris samples and incubated (60 minutes; 37° C.). Next, half of the samples were introduced to lambda exonuclease (15 units) and exonuclease VII (15 units) in lambda exonuclease buffer for a final volume of 50 uL, and were then incubated (60 minutes) to allow for the digestion of any unprotected DNA.

Agarose gel electrophoresis of a volume representing 100 ng equivalent starting amplicon sample was used for analysis. The results indicate that dCas9 protects the target nucleic acid with a Tris background (FIG. 8). Interestingly, the biotinylated dCas9 has greater fragment protection than the other Cas9/dCas9 complexes. For example, Lane 5 of FIG. 8 reveals that dCas9-guide RNA is unable to cut the amplicon, as represented by the lack of a band below 820 bp, but Lane 6 reveals that dCas9-guide RNA complex can protect a fragment slightly larger than the 275 bp cleavage product as represented by presence of the band at 275 bp after exposure to exonuclease; Lane 3 reveals that biotinylated dCas9-guide RNA complex is unable to cut the amplicon, as represented by the lack of a band below 820 bp, Lane 4 reveals that the biotinylated dCas9-guide RNA complex is not only able to protect the approximately 295 bp fragment after being exposed to exonuclease, it also affords a greater fragment protection than the other Cas9/dCas9 complexes by virtue of the intensity of the band at 275 bp; and Lanes 7 and 8 provide the Cas9-guide RNA complex cutting the amplicon as represented by the presence of fragments in Lane 7 at 545 bp and 275 bp and the protection of the 275 bp fragment as represented by the band at 275 bp in Lane 8.

The results of the data show that both Cas9 and dCas9 are capable of binding to and protecting target DNA suspended in a buffer solution (i.e., a non-plasma solution) under the guide of RNA. Furthermore, dCas activity can be modified (e.g., increased protection) by exposing the dCas to biotin.

### EXAMPLE 3

The protection activity of Cas9 in plasma is shown in this experiment. Results from this experiment indicate that Cas9 is capable of binding to and protecting the target nucleic acid in a plasma sample, and that surprisingly, dCas9 is unable to protect the target nucleic acid in the plasma sample. For example, Cas9 cleaves and protects target nucleic acid when exposed to both a plasma and a plasma free environment, while, dCas9 being able to protect a target nucleic acid in a plasma free environment, is unable to protect the target nucleic acid in plasma.

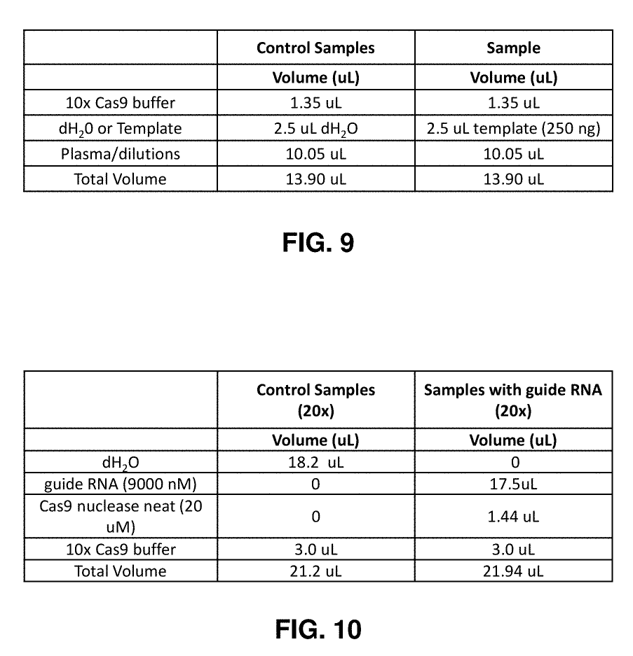

In this exemplary experiment, a control solution with DNA spike into Tris buffer and an experimental solution with DNA spiked into human plasma were used. The control solution (FIG. 9) was prepared using the same protocol outlined in Example 2, and the Cas9-complex formation in FIG. 7.

Human plasma samples were prepared by adding the 820 bp CFTR amplicon to the human plasma (FIG. 10) in a Streck tube. Cas9-guide RNA complexes for human plasma were prepared at higher concentrations than that of the control solution (FIG. 10). Particularly, 17.5 uL of guide RNA or biotinylated guide RNA (9000 nM), 1.44 uL of Cas9 nuclease or dCas9 nuclease (20 uM) and 3 uL of 10× Cas9 buffer were combined and then incubated (30 minutes, 25 C) to form the complex. Both Cas9 and dCas9 were used in combination with either guide RNA CFTR F2 or biotinylated guide RNA CFTR F2. Next, 1.06 uL of the complex was added to each of the human plasma samples and incubated (1 hour, 37 C) to allow for the Cas9 activity to occur.

After the cleavage reaction, half of the samples were incubated (1 hour, 37 C) with lambda exonuclease (15 units) and exonuclease VII (15 units) to degrade any unprotected DNA. The samples were then phenol-chloroform extracted, ethanol precipitated (with 10 ug glycogen added), and re-suspended in 10 mM Tris, pH 7.5, to achieve a final volume of 30 uL.

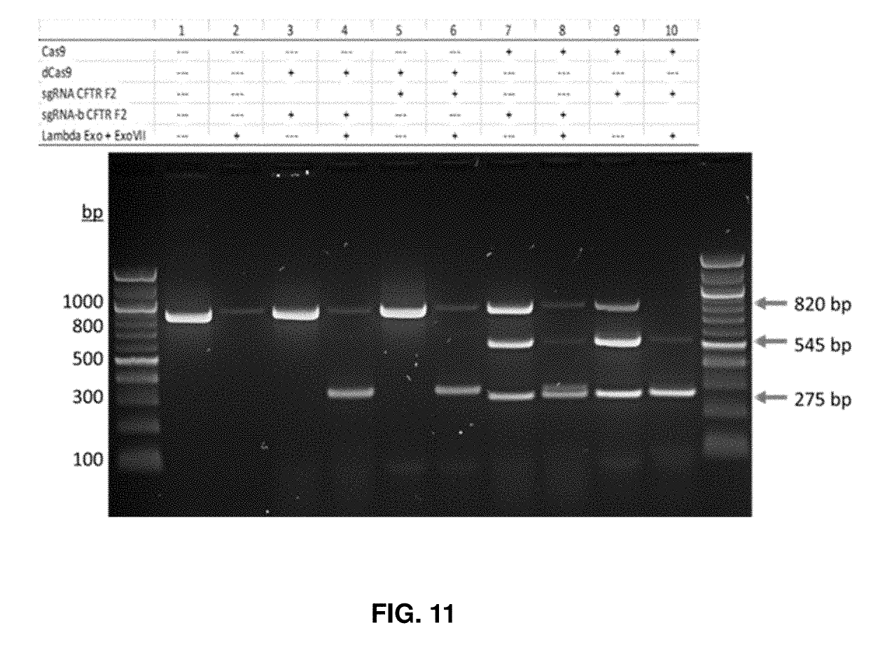

Agarose gel electrophoresis (with a volume representing 125 ng equivalent starting sample) was used for analysis. The results of treating an amplicon DNA with dCas9 or Cas9 complexed with either guide RNA CFTR F2 or biotinylated guide RNA CFTR F2 in Cas9 buffer (i.e., the control) indicate that Cas9 coupled with either guide RNA or biotinylated guide RNA is able to both cut and protect the target nucleic acid and dCas9 complexed with either guide RNA or biotinylated guide RNA is able to protect, but not cut the target nucleic acid (or slightly more than the target nucleic acid) (FIG. 11). For example, the bands present at 275 bp in Lanes 8 and 10 signify the protection of the amplicon by Cas9 and the bands present at 820 bp, 545 bp and 275 bp in Lanes 9 and 7 evidence the cutting of the amplicon by Cas9; the bands present at 275 bp in Lanes 4 and 6 evidence the protective effect of dCas9 and the lack of a band below 820 bp in Lanes 3 and 5 evidence dCas9's inability to cut the amplicon.

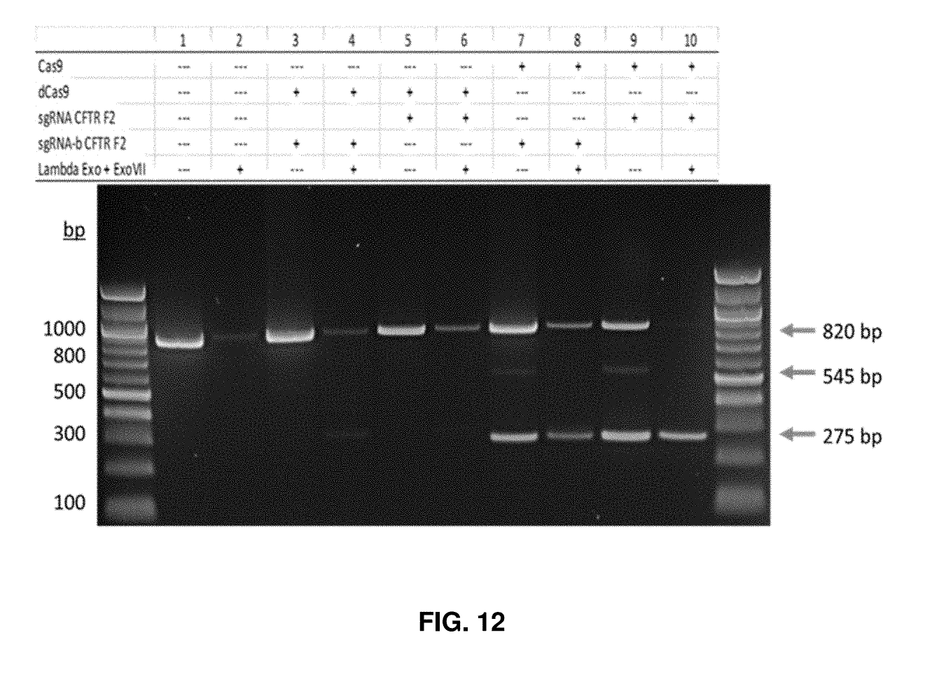

The results of treating an amplicon DNA with dCas9 or Cas9 complexed with either guide RNA CFTR F2 or biotinylated guide RNA CFTR F2 in human plasma indicate that Cas9 coupled with either guide RNA or biotinylated guide RNA is able to bind to, cut and protect the target nucleic acid directly in human plasma. Despite dCas9's abilities in non-plasma environment, dCas9 complexed with either guide RNA or biotinylated guide RNA is unable to protect target nucleic acid (FIG. 12). For example, Lanes 9 and 10 evidence that Cas9-sgRNA cuts the amplicon and protects the 275 bp fragment, even though the 545 bp cleavage fragment is present at a much lower intensity relative to the 275 bp fragment; Lanes 7 and 8 evidence that the biotinylated guide RNA has little effect on the activity of Cas9, but does seem to result in some additional protection in the absence of cutting for the Cas9-sgRNA complex, as evidence by the faint band in Lane 8 migrating slightly larger in size than the 275 bp band in Lane 7; Lanes 3-6 evidence that dCas9 complexed with either guide RNA or biotinylated guide RNA, does not protect the amplicon from digestion by the exonuclease in human plasma.

The results of these exemplary experiments provide that dCas9 is unable to protect target nucleic acid when introduced directly into a plasma sample. Importantly, this suggests an important mechanism for future work where dCas9 may not bind as tightly in plasma and is easier to displace during exonuclease treatment than the Cas9 complexes. As such, Cas9, and not dCas9, may be introduced directly into a plasma sample to protect and also detect target nucleic acid.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A method for enriching a sample, the method comprising:
obtaining a plasma sample comprising a target nucleic acid;
introducing Cas endonuclease to the plasma sample to bind to the target nucleic acid, wherein the Cas endonuclease is not dCas9; and
introducing an exonuclease to the plasma sample to digest unbound nucleic acid

2. The method of claim 1, wherein the Cas endonuclease is Cas9.

3. The method of claim 2, wherein the introduction step comprises introducing the Cas9 and guide RNA into the plasma sample and binding the Cas9 to ends of the target nucleic acid.

4. The method of claim 1, wherein the target nucleic acid comprises cDNA, cfDNA, or ctDNA.

5. The method of claim 1, further comprising isolating the target nucleic acid.

6. The method of claim 1, further comprising amplifying the target nucleic acid to yield amplicons.

7. The method of claim 6, further comprising sequencing the target nucleic acid to produce sequence reads and analyzing the sequence reads to provide genetic information of a subject.

8. The method of claim 1, further comprising analyzing the target nucleic acid to describe one or more variants in a subject.

9. The method of claim 7, wherein the target nucleic acid comprises a variant specific to a disease.

10. The method of claim 9, wherein the target nucleic acid is present at no more than about 0.01% of cell-free DNA in the plasma sample.

11. The method of claim 1, further comprising detecting the target nucleic acid.

12. The method of claim 11, wherein the detection step comprises hybridizing the target nucleic acid to a probe or to a primer for detection or amplification, or labelling the target nucleic acid with a detectable label.

13. The method of claim 12, wherein the detection step comprises connecting the Cas endonuclease-bound target nucleic acid to a particle or column and removing other components of the plasma sample.

14. The method of claim 13, wherein the particle comprises an agent that binds to at least one protein to form a particle-bound segment.

15. The method of claim 13, wherein the particle comprises magnetic or paramagnetic material and the detection step further comprises applying a magnetic field to separate the particle-bound segment from the other components.

16. The method of claim 11, wherein the detection step comprises applying the sample to a column.

17. The method of claim 16, wherein the Cas endonuclease-bound target nucleic acid is separated from unbound nucleic acid in the sample by size exclusion, ion exchange, or adsorption.

18. The method of claim 11, wherein the detection step comprises gel electrophoresis.

19. A method for enriching a sample, the method comprising:
obtaining a non-plasma sample comprising a target nucleic acid;
introducing dCas9 to the sample to bind to the target nucleic acid; and
introducing an exonuclease to the plasma sample to digest unbound nucleic acid.

20. A method for detecting a nucleic acid, the method comprising:
obtaining a bodily fluid sample comprising a target nucleic acid;
introducing Cas9, and not dCas9, directly to the sample to bind to and protect the target nucleic acid;
introducing an exonuclease to the sample to digest unbound nucleic acid; and
detecting at least one protected nucleic acid.

21. The method of claim 20, wherein the bodily fluid sample is plasma.

22. A method for detecting disease in a patient, the method comprising:
obtaining a bodily fluid sample comprising a target nucleic acid from a patient;
introducing Cas9, and not dCas9, directly to the sample to bind to and protect the target nucleic acid;
introducing an exonuclease to the sample to digest unbound nucleic acid; and
detecting at least one protected target nucleic acid.

23. The method of claim 22, wherein:
the bodily fluid sample is plasma, and detecting the target nucleic acid thereby detect the disease in the patient.
